(19) United States
(12) Patent Application Publication
(10) Pub. No.: US 2008/0154358 A1
(43) Pub. Date: Jun. 26, 2008

(54) Heart valve prosthesis

(76) Inventors: Geoffrey Tansley, Kegworth (GB); Seyed Mohammed Ali Mirnajafi Zadeh, Irvine, CA (US)

Correspondence Address: Seyed M. Mirnajafi Zadeh, 10 Clover, Irvine, CA, 92604, US

(21) Appl. No.: 11/878,953

(22) Filed: Jul. 28, 2007

(30) Foreign Application Priority Data
Jul. 28, 2006 (AU) 2006904107

(51) Int. Cl. A61F 2/24

(52) U.S. Cl. 623/2.14; 623/2.18

(57) ABSTRACT

A heart valve prosthesis 4 for human implantation fabricated from bovine pericardial material which is harvested from a region of incipient low calcium content or other material, wherein pre-forming and fixing of the leaflets 5 of the valve to create a rapid change in direction of the surface modifies the stresses within the material leading to a reduction of the peak tensile stress magnitude and concomitant in-use calcification thereby to increase longevity of the valve,

### FIELD OF INVENTION

The present invention relates to a prosthetic heart valve for use in human patients.

### BACKGROUND OF INVENTION

The number of heart patients is increasing and heart valve dysfunction is one of the most common problems in cardiovascular medicine. Heart valve prostheses have been used since 1965 and in many cases they are the only feasible treatment for patients with heart valve dysfunction. There are two main types of prosthetic heart valves: valves made from synthetic material known as mechanical valves; and valves made from biological tissue viz bioprosthetic heart valves. These valves are check valves which act to allow flow only in one direction through the valves through the action of flexing of cusps in bioprostheses or closure of flaps in mechanical valves.

Mechanical valves are durable but they are not very blood compatible and usually require extensive anticoagulation therapy for the duration of the implant. Whereas, bioprostheses are blood compatible but they do not usually last as long as mechanical valves in patients before failure. One category of mechanical valves is fabricated from polymeric material to have flexible cusps; these valves often are similar in configuration to bioprosthetic valves. The main advantage of bioprostheses over mechanical valves is their blood compatibility, and normally they are used in elderly patients who cannot cope with the medical complications associated with mechanical valves such as anti-coagulation therapy.

Bovine pericardial, porcine and human-homografts are the major categories of bioprosthetic heart valves. Bovine pericardial valves are made from bovine pericardium, which is the membrane that envelopes the bovine heart. The Second generation of bovine pericardial heart valves generally display better durability than their discontinued predecessors, and appear to be as durable as the best porcine valves. Another advantage of bovine pericardial heart valves is their amenability to design—they are not subjected to the anatomic restrictions associated with native porcine aortic valve geometry that porcine valves suffer from.

Calcification is known to be a major factor which contributes to the failure of bioprostheses and polymeric cusp valves. It has been previously suggested that high stress areas in the cusps of these valves are more likely to become calcified. Mechanical stress plays an important role in the calcification of these valves and their longevity.

It is an object of the present invention to address or ameliorate one or more of the abovementioned disadvantages in bioprostheses and polymeric cusp valves.

### BRIEF DESCRIPTION OF THE INVENTION

In one broad form of the invention there is provided a heart valve prosthesis for use in human patients comprising:

a cusp capable of functioning as a valve;

wherein the cusp is flexible;

wherein the heart valve prosthesis is constructed of at least a significant proportion of biological tissue;

wherein the cusp is fixed so as to retain residual stresses.

Preferably, the heart valve prosthesis wherein the residual stresses within the cusp reduce maximum tensile stresses within the cusp when the cusp is in an open position.

Preferably, the heart valve prosthesis wherein the residual stresses are introduced into a region of the cusp selected from the group consisting of a commissural region, a stent area and a belly region of the cusp. Preferably, the heart valve prosthesis wherein the cusp has a region angularly protruding from an outer edge of the cusp and wherein a surface of the cusp has a rapid change in direction so as to retain the residual stresses.

Preferably, the heart valve prosthesis wherein the biological tissue can include bovine pericardium tissue.

Preferably, the heart valve prosthesis wherein the biological tissue can include xeno-transplanted pericardial tissue.

Preferably, the heart valve prosthesis wherein the biological tissue can include xeno-transplanted valve tissue.

Preferably, the heart valve prosthesis wherein the biological tissue can include transplanted human valve or dura mater tissue.

Preferably, the heart valve prosthesis wherein the heart valve prosthesis includes three cusps.

Preferably, the heart valve prosthesis wherein the heart valve prosthesis is attached to a patient's circulatory system by a sewing ring.

Preferably, the heart valve prosthesis wherein the heart valve prosthesis can be attached to a patient's circulatory system by a stent device.

Preferably, the heart valve prosthesis wherein the bovine pericardium tissue is harvested so as to reduce innate calcium concentration present in the biological tissue.

Preferably, the heart valve prosthesis wherein the bovine pericardium tissue is selectively harvested from a predetermined region of a bovine pericardial sack.

Preferably, the heart valve prosthesis wherein the region of the bovine pericardial sack is determined by a region designated as position number 13 in FIG. 2 of the accompanying drawings.

Preferably, the heart valve prosthesis wherein the region of the bovine pericardial sack is positioned generally 75 mm away in both X & Y coordinates from a position where the bovine pericardial sac was attached to an apex region of a bovine heart, before detachment.

Preferably, the heart valve prosthesis wherein the biological tissue is fixed in glutaraldehyde.

In a further broad form of the invention there is provided a heart valve prosthesis for use in human patients comprising:

cusp capable of functioning as a valve;

wherein the cusp is flexible;

wherein the heart valve prosthesis is constructed of at least a significant proportion of biological tissue;

wherein the cusp includes a surface angularly protruding from an outer edge of the cusp;

whereby the cusp can open and close to allow a flow of blood in use.

Preferably, the heart valve prosthesis wherein geometry of the cusp reduces maximum tensile stresses within the cusp when in an open position.

Preferably, the heart valve prosthesis wherein the cusp is fixed so as to introduce residual stresses within the cusp when the cusp is in a closed position so as to reduce tensile stresses in the cusp when the cusp is in an open position.

Preferably, the heart valve prosthesis wherein the residual stresses are introduced into a region of the cusp selected from the group consisting of a commissural region, a stent area and a belly region of the cusp.

Preferably, the heart valve prosthesis wherein the biological tissue can include bovine pericardium tissue.

Preferably, the heart valve prosthesis wherein the biological tissue can include xeno-transplanted pericardial tissue.

Preferably, the heart valve prosthesis wherein the biological tissue can include xeno-transplanted valve tissue.

Preferably, the heart valve prosthesis wherein the biological tissue can include transplanted human valve tissue.

Preferably, the heart valve prosthesis wherein the heart valve prosthesis includes three cusps.

Preferably, the heart valve prosthesis wherein the heart valve prosthesis is attached to a patient's circulatory system by a sewing ring.

Preferably, the heart valve prosthesis wherein the heart valve prosthesis can be attached to a patient's circulatory system by a stent device.

Preferably, the heart valve prosthesis wherein the bovine pericardium tissue is harvested so as to reduce innate calcium concentration present in the biological tissue.

Preferably, the heart valve prosthesis wherein the bovine pericardium tissue is selectively harvested from a predetermined region of a bovine pericardial sack.

Preferably, the heart valve prosthesis wherein the region of the bovine pericardial sack is determined by a region designated as position number 13 in FIG. 2 of the accompanying drawings.

Preferably, the heart valve prosthesis wherein the region of the bovine pericardial sack is positioned generally 75 mm away in both X & Y coordinates from a position where the bovine pericardial sac was attached to an apex region of a bovine heart, before detachment.

Preferably, the heart valve prosthesis wherein the biological tissue is fixed in glutaraldehyde.

In a further broad form of the invention there is provided a heart valve prosthesis for use in human patients comprising:

a cusp capable of functioning as a valve;

wherein the cusp is flexible;

wherein the heart valve prosthesis is constructed of at least a significant proportion of biological tissue;

wherein the cusp can open and close to allow a flow of blood in use;

wherein the biological tissue is selectively harvested from tissue that is low in innate calcium concentration.

Preferably, the heart valve prosthesis wherein geometry of the cusp reduces maximum tensile stresses within the cusp when in an open position.

Preferably, the heart valve prosthesis wherein the cusp is fixed so as to introduce residual stresses within the cusp when the cusp is in a closed position so as to reduce tensile stresses in the cusp when the cusp is in an open position.

Preferably, the heart valve prosthesis wherein the residual stresses are introduced into a region of the cusp selecting from the group consisting of a commissural region, a stent area and a belly region of the cusp.

Preferably, the heart valve prosthesis wherein the biological tissue can include bovine pericardium tissue.

Preferably, the heart valve prosthesis wherein the biological tissue can include xeno-transplanted pericardial tissue.

Preferably, the heart valve prosthesis wherein the heart valve prosthesis includes three cusps.

Preferably, the heart valve prosthesis wherein the heart valve prosthesis is attached to a patient's circulatory system by a sewing ring.

Preferably, the heart valve prosthesis wherein the heart valve prosthesis can be attached to a patient's circulatory system by a stent device.

Preferably, the heart valve prosthesis wherein the pericardial tissue is selectively harvested from a predetermined region of a bovine pericardial sack.

Preferably, the heart valve prosthesis wherein the region of the bovine pericardial sack is determined by a region designated as position number 13 in FIG. 2 of the accompanying drawings.

Preferably, the heart valve prosthesis wherein the region of the bovine pericardial sack is positioned generally 75 mm away in both X & Y coordinates from a position where the bovine pericardial sac was attached to an apex region of a bovine heart, before detachment.

Preferably, the heart valve prosthesis wherein the biological tissue is fixed in glutaraldehyde.

In a further broad form of the invention there is provided a heart valve prosthesis for use in human patients comprising:

a cusp capable of functioning as a valve;

wherein the cusp is flexible;

wherein the heart valve prosthesis is constructed of at least a significant proportion of polymeric material;

wherein the cusp is formed so as to introduce residual stresses within the cusp when the cusp is in a partially open position, intermediate a completely closed position and a completely open position, so as to reduce tensile stresses in the cusp when the cusp is in a open position.

Preferably, the heart valve prosthesis wherein the introduced residual stresses within the cusp reduce maximum tensile stresses within the cusp when the cusp is in an open position.

Preferably, the heart valve prosthesis wherein the residual stresses are introduced into a region of the cusp selected from the group consisting of a commissural region, a stent area, a belly region of the cusp.

Preferably, the heart valve prosthesis wherein the cusp has a region angularly protruding from an outer edge of the cusp and wherein a surface of the cusp has a rapid change in direction so as to retain the residual stresses.

Preferably, the heart valve prosthesis wherein the heart valve prosthesis includes three cusps.

Preferably, the heart valve prosthesis wherein the heart valve prosthesis is attached to a patient's circulatory system by a sewing ring.

Preferably, the heart valve prosthesis wherein the heart valve prosthesis can be attached to a patient's circulatory system by a stent device.

In a further broad form of the invention there is provided a method of constructing a heart valve prosthesis for use in human patients comprising: forming a cusp capable of functioning as a valve; wherein the cusp is flexible; wherein the prosthesis is constructed of at least significant proportion of biological tissue; wherein the cusp is fixed so as to introduce residual stresses within the cusp.

Preferably, the method of constructing a heart valve prosthesis wherein geometry of the cusp reduces maximum tensile stresses within the cusp when in an open position.

Preferably, the method of constructing a heart valve prosthesis wherein the residual stresses are introduced into a region of the cusp selected from the group consisting of a commissural region, a stent area and a belly region of the cusp.

Preferably, the method of constructing a heart valve prosthesis wherein the cusp has a region angularly protruding from an outer edge of the cusp and wherein a surface of the cusp has a rapid change in direction so as to retain the residual stresses.

Preferably, the method of constructing a heart valve prosthesis wherein the biological tissue can include bovine pericardium tissue.

Preferably, the method of constructing a heart valve prosthesis wherein the biological tissue can include xeno-transplanted pericardium tissue.

Preferably, the method of constructing a heart valve prosthesis wherein the heart valve prosthesis includes three cusps.

Preferably, the method of constructing a heart valve prosthesis wherein the heart valve prosthesis is attached to a patient's circulatory system by a sewing ring.

Preferably, the method of constructing a heart valve prosthesis wherein the heart valve prosthesis can be attached to a patient's circulatory system by a stent region.

Preferably, the method of constructing a heart valve prosthesis wherein the bovine pericardium tissue is harvested so as to reduce innate calcium concentration present in the bovine pericardium tissue.

Preferably, the method of constructing a heart valve prosthesis wherein the bovine pericardium tissue is selectively harvested from a predetermined region of a bovine pericardial sack.

Preferably, the method of constructing a heart valve prosthesis wherein the region of the bovine pericardial sack is determined by a region designated as position number 13 in FIG. 2 of the accompanying drawings.

Preferably, the method of constructing a heart valve prosthesis wherein the region of the bovine pericardial sack is positioned generally 75 mm away in both X & Y coordinates from a position where the bovine pericardial sac was attached to an apex region of a bovine heart, before detachment.

Preferably, the method of constructing a heart valve prosthesis wherein the biological tissue is fixed in glutaraldehyde. In a further broad form of the invention there is provided a method of constructing a heart valve prosthesis for use in human patients comprising: forming a cusp capable of functioning as a valve; wherein the cusp is flexible; wherein the prosthesis is constructed of at least a significant proportion of polymeric material; wherein the cusp is manufactured so as to introduce residual stresses within the cusp.

Preferably, the method of constructing a heart valve prosthesis wherein geometry of the cusp reduces maximum tensile stresses within the cusp when in an open position.

Preferably, the method of constructing a heart valve prosthesis wherein the residual stresses are introduced into a region of the cusp selected from the group consisting of a commissural region, a stent area and a belly region of the cusp.

Preferably, the method of constructing a heart valve prosthesis wherein the cusp has a region angularly protruding from an outer edge of the cusp and wherein a surface of the cusp has a rapid change in direction so as to retain the residual stresses.

Preferably, the method of constructing a heart valve prosthesis wherein the prosthesis can include bovine pericardium tissue.

Preferably, the method of constructing a heart valve prosthesis wherein the prosthesis can include xeno-transplanted pericardium tissue.

Preferably, the method of constructing a heart valve prosthesis wherein the heart valve prosthesis includes three cusps.

Preferably, the method of constructing a heart valve prosthesis wherein the heart valve prosthesis is attached to a patient's circulatory system, by a sewing ring.

Preferably, the method of constructing a heart valve prosthesis wherein the heart valve prosthesis can be attached to a patient's circulatory system by a stent region.

Preferably, the method of constructing a heart valve prosthesis wherein the bovine pericardium tissue is harvested so as to reduce innate calcium concentration present in the bovine pericardium tissue.

Preferably, the method of constructing a heart valve prosthesis wherein the bovine pericardium tissue is selectively harvested from a predetermined region of a bovine pericardial sack.

Preferably, the method of constructing a heart valve prosthesis wherein the region of the bovine pericardial sack is determined by a region designated as position 13 in FIG. 2 of the accompanying drawings.

Preferably, the method of constructing a heart valve prosthesis wherein the region of the bovine pericardial sack is positioned generally 75 mm away in both X & Y coordinates from a position where the bovine pericardial sac was attached to an apex region of a bovine heart, before detachment.

Preferably, the method of constructing a heart valve prosthesis wherein the biological tissue is fixed in glutaraldehyde.

In a further broad form of the invention there is provided a method for construction of a valve member for use in a substantially one-way heart valve; the member including a lip region and an attachment region, the attachment region being substantially fixed to the valve, the method comprising the step of:

fixing a change of direction in the member during manufacture;

wherein the member adopts a substantially unstressed state in a position intermediate a first plane of alignment and a second plane of alignment;

wherein the member can flex with respect to the attachment region;

wherein the member can move in alignment between the first plane of alignment and the second plane of alignment.

Preferably, the method wherein the method can produce a deformity in the member.

Preferably, the method wherein the member can include a cusp.

### BRIEF DESCRIPTION OF DRAWINGS

Embodiments of the present invention will now be described with reference to the drawings in which:

FIG. 1 is a photograph of a complete bovine pericardium sack which has been excised and laid flat and which shows anatomical landmarks. A square section of the pericardium, denoted ABCD, has been cut out of the pericardial sheet

FIG. 2 is a diagram of the cut-out section, ABCD, of pericardium from the previous FIG. 1. This cut-out section has been divided into regions as indicated;

FIG. 3 is an isometric view of a bioprosthetic heart valve showing a distal perspective of the arrangement of three cusps in a preferred arrangement;

FIG. 4 is an isometric view of the heart valve showing the proximal surfaces of the cusps.

FIG. 5 shows an isometric view of a section through the bioprosthetic heart valve. Particularly, this Figure shows the shape of one of the cusps at its centreline.

FIG. 6 tabulates the approximate shape of the centreline of the cusps in prior-art and in the new invention and shows corresponding stress profiles within the cusps.

FIG. 7 depicts a schematic section of a cusp and stenting device viewed essentially at the centreline of the cusp in a prior-art valve in 7A, and in the present invention in 7B.

### DETAILED DESCRIPTION OF EMBODIMENTS

The most preferred embodiment of the present invention is a heart valve prosthesis for use in human patients; wherein the prosthesis is constructed of a significant proportion of biological tissue; wherein the prosthesis includes at least one cusp capable of functioning as a valve; wherein the valve includes a flexible cusp and a surface angularly protruding from an outer edge of the cusp and wherein the cusp can open and close to allow a flow of blood only in one direction, in use.

In a preferred embodiment the heart valve prosthesis includes three cusps that are attached to a stenting device. The number of cusps can be altered where appropriate. The heart valve prosthesis can be attached to the patient's heart or circulatory system by way of stenting, bioglue, and/or sewing. A sewing ring and stent might be provided to aid sewing, but in an alternative embodiment the sewing ring might be omitted.

Preferably the cusps are constructed from bovine heart tissue, in particular the bovine pericardial sac. Please note that other biological materials can be used including but not limited to human homografts, human dura mater, and porcine tissue. The cusps can also be made from polymeric material.

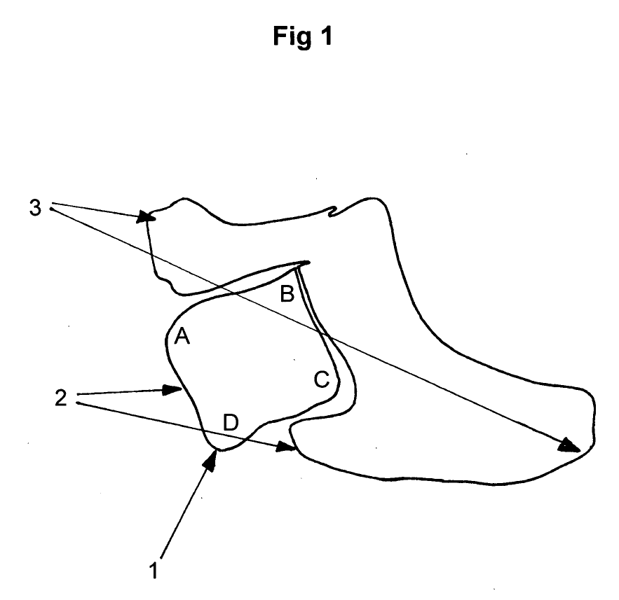

In FIG. 1, a complete bovine pericardial sac or bovine pericardium is shown and anatomical landmarks are illustrated. This figure shows the sac once it is removed from the bovine heart and laid flat. A marked rectangular region of the bovine pericardial sac, which is delineated by A, B, C, & D, marks the preferred region from which a cusp can be constructed. The location at which the pericardium was contacting the apex area of the heart is marked by position 1. Cut lines 2 show the locations at which the pericardium was cut to remove it from its attachment to the middle of the left ventricle of the heart. Position 3 marks the location at which the pericardium was cut away from an aortic attachment.

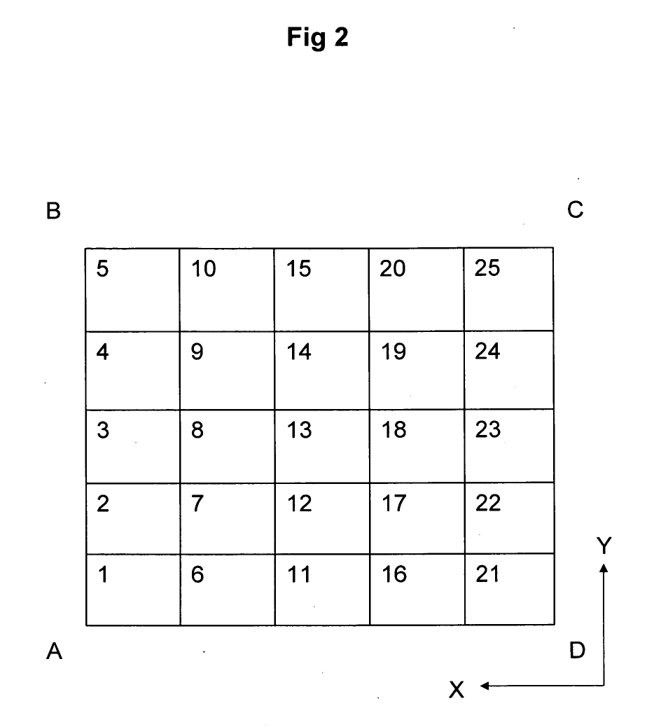

FIG. 2 shows an inset of the delineated region A, B, C & D. This region is further divided into smaller numbered regions called ‘Position Numbers’ which are numerically marked in this diagram.

There is a variation of calcium levels within different areas of the bovine pericardium, and that variation is consistent between different bovine pericardia. It has been determined scientifically that the smaller region marked Position Number 13 is the most preferred area from which a cusp can be constructed. Position Number 13 has been found to have a relatively low amount of calcium compared with the remainder of the bovine pericardium. Position Number 13 also has shown the lowest coefficient of variation in a study of multiple pericardia.

The initial level of calcium in bovine pericardium is important in reducing the likelihood of initiating calcification of the preferred heart valve prosthesis. Therefore reducing the initial level of calcium within the material from which the preferred heart valve prosthesis is constructed can significantly increase the longevity of the bioprosthesis.

Typically, the region ABCD in FIG. 2 can be a flat square of approximately 150 mm×150 mm constructed from bovine pericardium. Typically, the smaller numbered regions featured in FIG. 2 can be 30 mm×30 mm. Location of the corners A, B, C, & D are consistent throughout FIGS. 1 & 2, where corner D in FIG. 1 represents the bovine pericardium in the apex area. The region ABCD can be the preferred area from which to make the cusps of bioprosthesis as the thickness of bovine pericardium has relatively: less variation; fewer nodes; and less fat tissue. It has also been found that position 13 shown in FIG. 2 is approximately at a point 75 mm away from the apex in respect of both the X & Y coordinates within the plane of the tissue sheet.

Please note that the initial calcium level within fresh bovine pericardium is generally between the ranges 0-5 μg/mg tissue. It has also been shown that the tissue at position number 13 in FIG. 2 can generally be the most suitable for use in the construction of heart valve prosthesis. This can be generally due to the comparatively low concentration of calcium in the tissue.

It has also been scientifically shown that there is a direct relationship between the mechanical stress experienced by a heart valve prosthesis and the amount of calcium adsorbed by the cusps of the preferred prosthesis. It has been demonstrated that regions of the heart valve prosthesis which experience higher levels of mechanical stress are more likely to absorb calcium. The amount of calcium absorbed is directly proportional to the amount of tensile stress applied to the tissue.

Calcium absorption plays a significant role in calcification of the heart valve prosthesis, because calcium absorption leads directly to this calcification. It is noted that calcification can generally lead to impedance or a reduction in performance for heart valve prosthesis; calcification should be avoided. In the worst cases of calcification, the cusps of the heart valve prosthesis can become rigid and/or can break or tear.

As mechanical stressing, particularly tensile stresses, in the bovine pericardium can act to increase calcium absorption in to the tissue; mechanical stress also should be reduced. It can be preferred to attempt to reduce the level of mechanical stress experienced by the tissue forming part of the preferred embodiments.

Attempts to reduce mechanical stress experienced, particularly by the cusps of the preferred embodiment, can lead to significant increases in the longevity of the cusps and/or heart valve prosthesis. Therefore it is desirable to additionally alter or modify the design of heart valve prostheses to reduce stress to its component parts in particular the cusps.

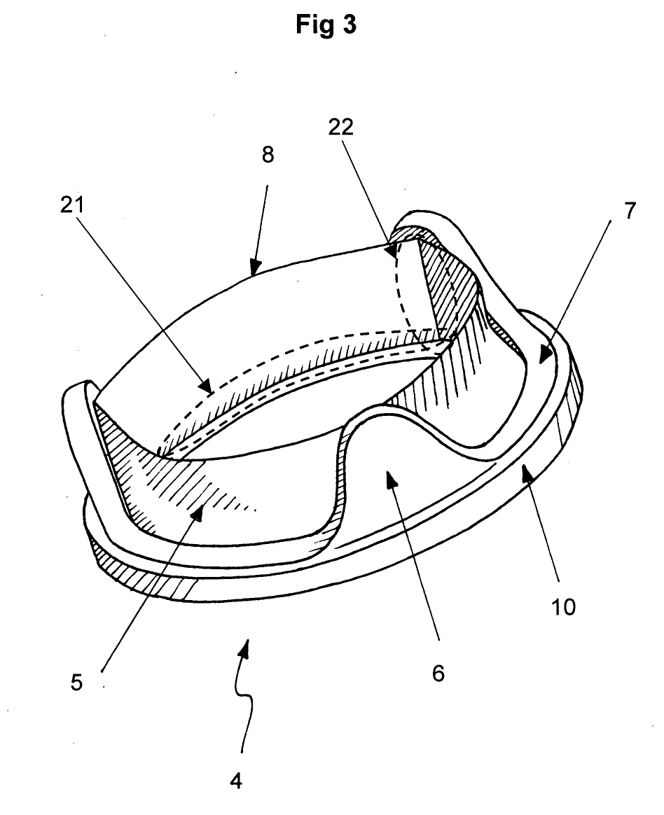

It has also been scientifically shown that the maximum stresses in a heart valve prosthesis generally occur in the commissural area 22 of a cusp shown in FIG. 3. It is therefore desirable to reduce tensile stress experienced by the heart valve prosthesis by modification to the design and shape of a cusp of the heart valve.

Please note that glutaraldehyde fixation or similar fixation is necessary for all bioprostheses to prevent the implant from being rejected by the recipient's immune system after implantation.

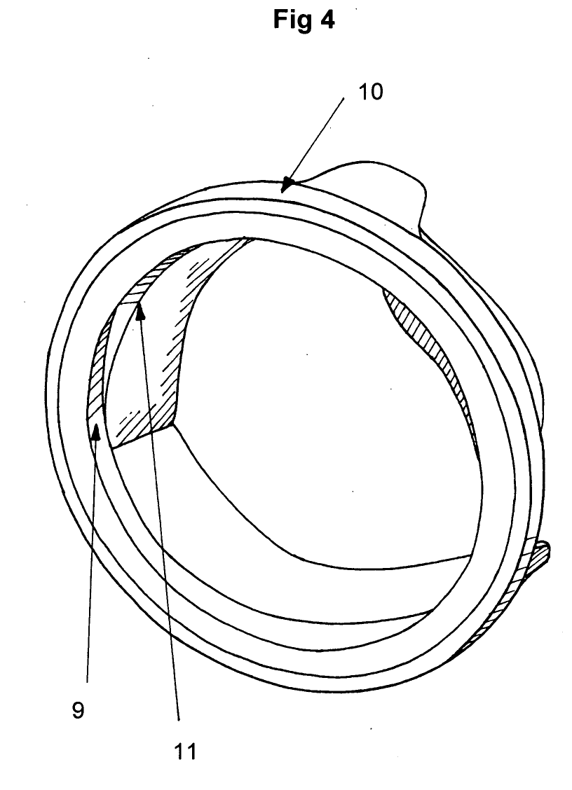

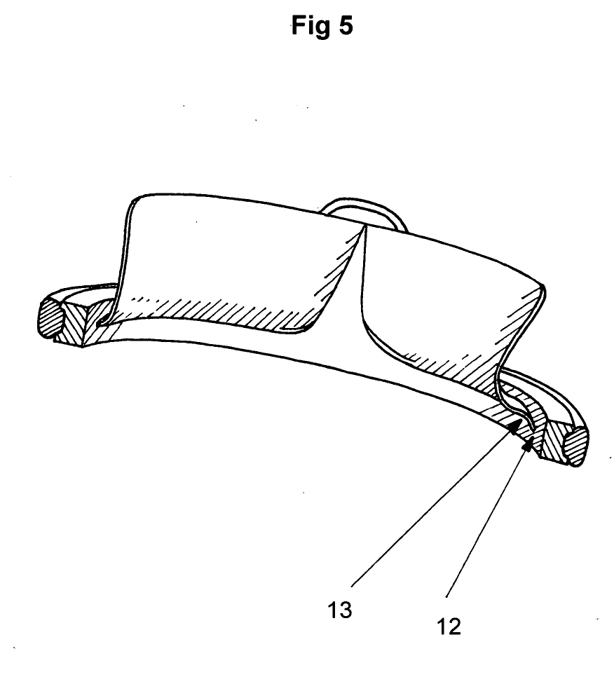

A preferred embodiment of the shape and configuration of the preferred cusp forming part of preferred heart valve prosthesis 4 is shown in FIG. 3. The cusp 5 is sewn to a stenting device 7 at the stenting posts 6 and along a sewing line 9, as seen in FIG. 4. As shown in FIGS. 3 and 4, the stenting device can in turn be affixed to a sewing ring 10 which can be sewn to the annulus in the heart whence the natural valve was removed. The lips 8 of the cusps are free to move in response to the flow and pressure of the blood, such that on contraction (systole) of the heart, the lips 8 open and allow passage of the blood. This defines the open phase for the cusps 5 of the heart valve 4. The material suspended between the stents is not taught but reasonably loose to allow flexure and the material will sag and form a belly 21 to each cusp in the open phase. On relaxation (diastole) of the heart, the pressure gradient across the heart valve 4 causes the lips 8 to come together and prevent blood flow back into the heart. This defines the closed phase of the cusps 5 of the heart valve 4. The period between the open phase and the closed phase wherein the lips start to come together is defined as the closing phase. The opening phase is the period wherein the lips separate as the valve moves towards its open phase. A set region 12 close to the sewing line 9 in the shown embodiment in FIG. 5 can extend angularly away from the sewing line 9. The set region 12 is bounded by a ridge formation 13 which comprises a distinct change of angle of the surface of the cusp. This change of angle is fixed into the cusps with gluteraldahyde during the manufacturing process. Preferably, this change of angle is generally between 0° and 90°.

The configuration of the cusp 5 shown in this embodiment depicts a possible shape which will minimise tensile stress experienced by movement or bending of the cusp 5 in use. Thereby, this shape and/or configuration can lead to a reduction of calcification of a heart valve prosthesis constructed from this shape of cusp. Additionally, the shape of cusp can significantly increase life or longevity of the cusp 5, and the heart valve prosthesis 4 which this cusp can be incorporated within.

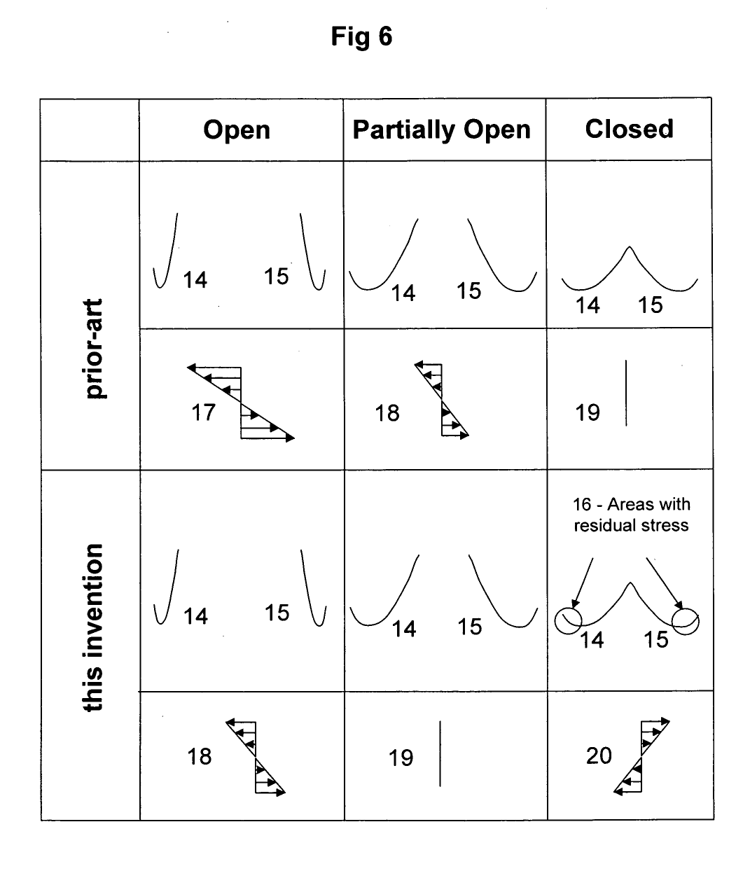

To attain residual stress in the cusp and also the shape of the cusp 5 and its concomitant benefits of reduced calcification and improved longevity, the cusp 5 must be bent or deformed, in the opposite direction to that which will occur when in use, to create the ridge formation 13, and cross-linked using glutaraldehyde or other fixation method prior to use. In this way residual stresses are generated such that in use, the peak tensile stresses which occur in the open valve are decreased. Furthermore, the phase within the cardiac cycle at which the resting leaflet displaying zero stress occurs can be altered from the closed phase to the opening phase which can lead to a reduction of the maximal stresses in the fully open valve and thus further reduce the likelihood of calcification. FIG. 6 compares the stresses in the cusps of a prior-art valve and in the present invention; the cross-sectional shapes of the cusps 14 and 15 are shown in the open, partially open and closed positions. The arrow diagrams below each schematic represent: 17 the high stresses in an open prior-art valve; 18 reduced stresses in the prior-art valve cusps as the valve opens or closes; 19 zero or low stresses on the cusps of a closed prior art valve. These stresses can be compared with: 18 reduced stresses in the open cusps of the present invention; 19 zero or minimal stresses in the cusps of the present invention whilst partially open and; 20 reduced stresses or opposite sense in the closed cusps of the present invention. These stress states and in particular the residual stresses in the cusps are invoked by the aforementioned gluteraldyhyde fixation of the cusps whilst bent or manipulated into a suitable shape.

This configuration of the cusp allows the maximum stress of the open phase to be considerably less than the maximum stress of the open phase of prior art heart valve bioprostheses. However the less significant maximal tensile stress of the closed valve of the present embodiment can be more (112.9 kPa) in some areas (16) than the comparable stress experienced by the prior art designs (42.5 kPa). The maximum tensile stress that can occur in the present embodiment is reduced (316.7 kPa) in the critical open valve when compared with the maximum tensile stress (363.7 kPa) in the cusps of prior art designs when open. Thus the maximum stress occurring anywhere in the cycle is reduced at a cost of an increase in stresses in the less important closed phase of the valve.

Additionally, we note that porcine tissue material can be used in the construction of a preferred embodiment. Porcine tissue has the advantage that the tissue does not have to be resized but it would need to be re-shaped. Residual stress can be produced in porcine tissue by bending leaflets of the valve in the required directions before glutaraldehyde fixation or other cross-linking method.

Bovine tissue can also be preferred because it is generally more resistant to calcification than porcine tissue.

A preferred embodiment of the present invention can also exclude a stenting device allowing attachment of the prosthesis directly into the annulus in the heart whence the patient's natural heart valve was removed.

Yet another preferred embodiment of the present invention can also include valves made from polymeric material, the cusps of which are given a permanent bend similar to that of the ridge formation 13 of the bioprostheses which will introduce residual stresses. These residual stresses will act to reduce the level of tensile stresses attained in the open phase of the cardiac cycle and thus reduce the likelihood of calcification and concomitant failure of the polymeric cusps.

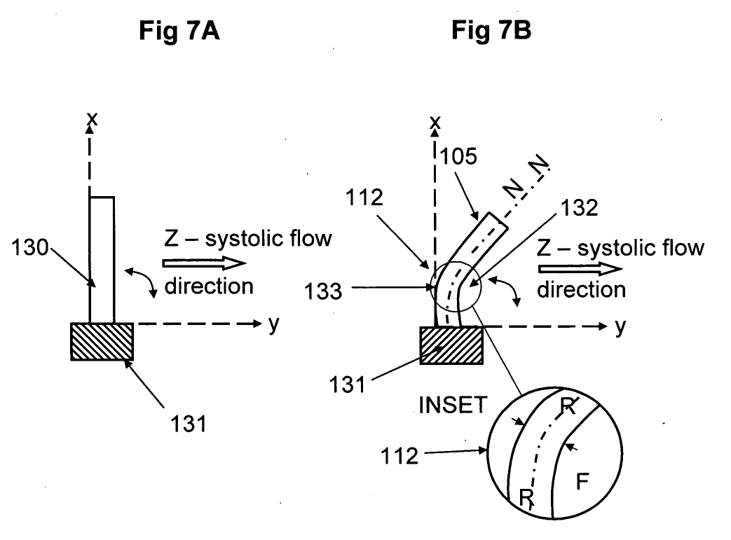

In use with reference to FIG. 7 there is illustrated a further generalized embodiment of a heart valve arrangement in accordance with the present invention illustrated for comparison against a typical prior art arrangement.

More specifically FIG. 7A illustrates a generalized version of a prior art valve arrangement whilst FIG. 7B illustrates a generalized embodiment of the present invention. Like components are numbered as for previous embodiments except in the one-hundreds series so, for example, leaflet or cusp 5 becomes leaflet or cusp 105 in this embodiment.

Initially with reference to FIG. 7A there is illustrated a prior art valve leaflet or cusp 130 which may comprise either a biological or non-biological material. The cusp 130 is viewed in cross section and in the manner in which it may be utilised to form the operational part of a one-way valve structure. The cusp 130 is anchored or otherwise attached to a reference portion 131 of the valve structure (not shown) in such a way that its unstressed natural orientation lies along axis OX as illustrated. In use the cusp 130 can bend about an axis substantially lined through reference portion 131 so as to ultimately align with axis OY and positions continuously in between. Typically the unstressed position illustrated in FIG. 7A corresponds to a closed valve position in accordance with flow through the valve being in the direction of arrow Z as illustrated.

Because cusp 130 is fixedly attached to reference portion 131 stresses will occur in the region of flexure of the cusp 130 as it moves to align with axis OY. Typically for one way valve-type applications the angular separation between axis OX and axis OY is up to approximately 90 degrees.

With reference to FIG. 7B there is illustrated cusp 105 in accordance with an embodiment of the present invention which, similarly, is anchored to reference portion 131 of a valve structure (not shown). As for the prior art arrangement of FIG. 7A, in use, the leaflet or cusp 105 will operate between a substantially closed position when aligned with axis OX and a substantially open position when aligned with axis OY as referenced against a one-way flow in the direction of arrow Z.

In the preferred embodiment of the present invention a set region 112 is introduced into cusp 105 so as to bias cusp 105 when in an unstressed state to an angular position intermediate axes OX and OY.

As illustrated in the inset the set region 112 is typically selected to be associated with a region in which stress will vary substantially as the cusp 105 moves angularly with respect to reference portion 131 and axes OX and OY. In the case of a curved set region 112 which align with a region or flexure it can be expected that a compressive force or stress of positive magnitude F can be experienced substantially at right-angles to the line RR of set region 112. As cusp 105 moves from its neutral alignment along axis NN in the direction of axis OY. Conversely as cusp 105 moves from its neutral alignment axis NN in the direction of axis OX forces of a negative magnitude (−F) will be experienced substantially at right-angles to the alignment RR of the inset to FIG. 7B. This is to be contrasted with the arrangement of FIG. 7A wherein comparable forces or stresses F will be of one sign only.

In practice set region 112 will comprise a bend or associated change in direction or alignment of cusp 105, which direction or change in alignment is set during manufacture of the cusp 105 so as to define a natural substantially unstressed position to which the cusp 105 returns naturally in the absence of fluid forces, in use, acting upon it.

Broadly speaking the set region is selected so that the substantially unstressed or least stressed state of cusp 105 will, in use, lie somewhere between axes OX and OY with reference to referenced portion 131 and, in use, will oscillate thereabout so as to reach substantially positive or negative stress values F as cusp alignment approaches one or other of axes OX and OY but in substantially all cases being of a magnitude less than typically expected for the maximum magnitude of stress force exhibited by leaflet or cusp 130 of the prior art arrangement of FIG. 7A.

The principles described above can be applied to leaflet or cusp structures constructed from either biological tissue or synthetic materials. Particular examples have been given in the earlier described embodiments for both types of materials.

### In Use

Residual stresses can be introduced into the cusp in at least one of a commissural region, a stent area and a belly region of the cusp. Additionally, the surface of the cusp can be formed so as to include a rapid change in direction of the surface so as to introduce residual stresses into the cusp. Additional embodiments can also be contemplated so as to introduce residual stresses into the cusp, so as to minimize tensile stresses.

Various additional modifications and variations are possible within the scope of the foregoing specification and accompanying drawings without departing from the scope of the invention.

1. A heart valve prosthesis for use in human patients comprising:
at least one cusp capable of functioning as a valve;
wherein the cusp is flexible;
wherein the heart valve prosthesis is constructed of at least a significant proportion of biological tissue;
wherein the cusp is fixed so as to retain residual stresses.

2. The heart valve prosthesis of claim 1 wherein the residual stresses within the cusp reduce maximum tensile stresses within the cusp when the cusp is in an open position.

3. The heart prosthesis of claim 2 wherein the residual stresses are introduced into a region of the cusp selected from the group consisting of a commissural region, a stent area and a belly region of the cusp.

4. The heart valve prosthesis of claim 3 wherein the cusp has a region angularly protruding from an outer edge of the cusp and wherein a surface of the cusp has a rapid change in direction so as to retain the residual stresses.

5. The heart valve prosthesis of claim 1 wherein the biological tissue can include bovine pericardium tissue.

6. The heart valve prosthesis of claim 1 wherein the biological tissue can include xeno-transplanted pericardial tissue.

7. The heart valve prosthesis of claim 1 wherein the biological tissue can include xeno-transplanted valve tissue.

8. The heart valve prosthesis of claim 1 wherein the biological tissue can include transplanted human valve or dura mater tissue.

9. The heart valve prosthesis of claim 1 wherein the heart valve prosthesis is attached to a patient's circulatory system by a sewing ring.

10. The heart valve prosthesis of claim 1 wherein the heart valve prosthesis can be attached to a patient's circulatory system by a stent device.

11. The heart valve prosthesis of claim 1 wherein the bovine pericardium tissue is selectively harvested from a predetermined region of a bovine pericardial sack so as to reduce innate calcium concentration present in the biological tissue.

12. The heart valve prosthesis of claim 1 wherein the biological tissue is fixed in glutaraldehyde.

13. A heart valve prosthesis for use in human patients comprising:
at least one cusp capable of functioning as a valve;
wherein the cusp is flexible;
wherein the heart valve prosthesis is constructed of at least a significant proportion of biological tissue;
wherein the cusp can open and close to allow a flow of blood in use;
wherein the biological tissue is selectively harvested from tissue that is low in innate calcium concentration.

14. The heart valve prosthesis of claim 13 wherein the region of the bovine pericardial sack is determined by a region designated at position number 13 in FIG. 2 of the accompanying drawings.

15. The heart valve prosthesis of claim 14 wherein the cusp is fixed so as to introduce residual stresses within the cusp when the cusp is in a closed position so as to reduce tensile stresses in the cusp when the cusp is in an open position.

16. The heart valve prosthesis of claim 15 wherein the residual stresses are introduced into a region of the cusp selected from the group consisting of a commissural region, a stent area and a belly region of the cusp.

17. The heart valve prosthesis of claim 13 wherein the biological tissue can include bovine pericardium tissue.

18. A heart valve prosthesis for use in human patients comprising:
at least one cusp capable of functioning as a valve;
wherein the cusp is flexible;
wherein the heart valve prosthesis is constructed of at least a significant proportion of polymeric material;
wherein the cusp is formed so as to introduce residual stresses within the cusp when the cusp is in a partially open position, so as to reduce tensile stresses in the cusp when the cusp is in an open position.

19. The heart valve prosthesis of claim 18 wherein the residual stresses are introduced into a region of the cusp selected from the group consisting of a commissural region, a stent area, a belly region of the cusp.

20. The heart valve prosthesis of claim 18 wherein the cusp has a region angularly protruding from an outer edge of the cusp and wherein a surface of the cusp has a rapid change in direction so as to retain the residual stresses.
